(19) United States
(12) Patent Application Publication
(10) Pub. No.: US 2025/0312117 A1
(43) Pub. Date: Oct. 9, 2025

(54) SURGICAL ROBOT, MASTER MANIPULATOR ARM CAPABLE OF MAINTAINING POSE, AND JOINT TRANSMISSION STRUCTURE OF MANIPULATOR ARM

(71) Applicant: CHONGQING JINSHAN MEDICAL ROBOTICS CO., LTD., Chongqing (CN)

(72) Inventors: Jihui TANG, Chongqing (CN); Liao WANG, Chongqing (CN); Yu HUANG, Chongqing (CN)

(73) Assignee: CHONGQING JINSHAN MEDICAL ROBOTICS CO., LTD., Chongqing (CN)

(21) Appl. No.: 18/865,308

(22) PCT Filed: May 27, 2022
(86) PCT No.: PCT/CN2022/095611

§ 371 Date: Nov. 12, 2024

(30) Foreign Application Priority Data
May 12, 2022 (CN) 202210514097.2
May 12, 2022 (CN) 202210514098.7

(51) Int. Cl. A61B 34/00; A61B 17/00; A61B 34/37; A61B 90/00; B25J 3/00; B25J 9/10

(52) U.S. Cl. CPC A61B 34/70; A61B 34/37; B25J 3/00; B25J 9/1065; A61B 2017/00199; A61B 2090/066; A61B 2090/067

(57) ABSTRACT

A master manipulator arm capable of maintaining pose, a joint transmission structure of a manipulator arm, and a surgical robot are provided. In the master manipulator arm, multiple first linkage joints on a front linkage use a joint module to adjust the angle and displacement, multiple second linkage joints of a rear linkage use a transmission gear pair to perform angle and displacement transmission. The mounting structure of the rear linkage can be more compact. A doctor controls a master tool to transmit a surgical operation from the manipulator arm to a surgical instrument. An arm joint uses of the transmission gear pair to perform angle and displacement transmission.

The present application claims the priorities to Chinese Patent Applications No. 202210514097.2, titled “SURGICAL ROBOT AND MASTER MANIPULATOR ARM CAPABLE OF MAINTAINING POSE”, and No. CN202210514098.7, titled “SURGICAL ROBOT AND MASTER MANIPULATOR ARM CAPABLE OF MAINTAINING POSE”, both filed with the China National Intellectual Property Administration on May 12, 2022, the entire disclosures of which are incorporated herein by reference.

### FIELD

The present application relates to the technical field of surgical robots, and in particular to a surgical robot, a master manipulator arm capable of maintaining pose, and a joint transmission structure of a manipulator arm.

### BACKGROUND

With the application and development of robot technology, the effects of surgical robots in clinical practice are increasingly valued by people. In order to achieve good real-time status feedback to cope with complex situations during surgery, master-slave manipulator arms are generally applied in medical robot systems for surgery. The operator drives a master manipulator arm to move by a master tool, and the movement information of the joints of the master manipulator arm is mapped to a slave manipulator arm to drive the slave manipulator arm to make corresponding movements.

However, the conventional master manipulator arms are diverse, which have their own advantages and disadvantages in terms of performance and cannot meet the diverse requirements of medical robots for practical application environments. For example, in case of a power outage, the postural stability of the master manipulator arm is directly related to the operation safety of the medical surgical robot.

It should be noted herein that the technical contents in this section are intended to help those skilled in the art to understand the present invention, and does not necessarily constitute the prior art.

### SUMMARY

In view of this, an object of the present application is to provide a master manipulator arm capable of maintaining pose and a joint transmission structure of a manipulator arm, to improve the operation safety of the master manipulator arm. A surgical robot is further provided in the present application.

In order to achieve the above object, the technical solutions are provided in the present application as follow.

A master manipulator arm capable of maintaining pose includes a front linkage proximate to a fixed mounting side and a rear linkage proximate to a master tool side, where joint modules configured to adjust an angle and a displacement are arranged at first linkage joints of the front linkage, and transmission gear pairs configured to adjust an angle and a displacement are arranged at second linkage joints of the rear linkage.

Preferably, in the master manipulator arm capable of maintaining pose, the front linkage includes a first-axis linkage, a second-axis linkage, a third-axis linkage, and a fourth-axis linkage; and
a first joint module is arranged between the first-axis linkage and the second-axis linkage, a second joint module is arranged between the second-axis linkage and the third-axis linkage, and a third joint module is arranged between the third-axis linkage and the fourth-axis linkage.

Preferably, in the master manipulator arm capable of maintaining pose, the rear linkage includes a fourth-axis linkage, a fifth-axis linkage, a sixth-axis linkage and a seventh-axis linkage, where a master tool is arranged at a free end of the seventh-axis linkage;
the fourth-axis linkage and the fifth-axis linkage are in transmission through a first gear pair, and a first reduction motor is provided in the first gear pair;
the fifth-axis linkage and the sixth-axis linkage are in transmission through a second gear pair, a second reduction motor is provided in the second gear pair;
the sixth-axis linkage and the seventh-axis linkage are in transmission through a third gear pair, and a fourth reduction motor is provided in the third gear pair; and
the seventh-axis linkage and the master tool are in transmission through a fourth gear pair, and a fourth reduction motor is provided in the fourth gear pair.

Preferably, in the master manipulator arm capable of maintaining pose, the first gear pair, the second gear pair, the third gear pair, and the fourth gear pair are all bevel gear pairs.

Preferably, in the master manipulator arm capable of maintaining pose, an angle sensor and a displacement sensor are provided in each of the first joint module, the second joint module, and the third joint module, to record movement velocities and positions of downstream shafts and upload the movement velocities and the positions to a control system.

Preferably, in the master manipulator arm capable of maintaining pose, a force sensor, configured for measuring a torque of a joint movement between the corresponding linkages, is provided in each of the front linkage and the rear linkage.

Preferably, in the master manipulator arm capable of maintaining pose, each of the transmission gear pairs includes a reduction motor assembly arranged inside an upstream linkage, an axial direction of the reduction motor assembly is perpendicular to an axis of a joint located at a power output end of the reduction motor assembly; and
the power output end of the reduction motor assembly is provided with a bevel gear pair with orthogonal axes, which is configured to drive a downstream linkage being in transmission cooperation with the power output end of the reduction motor assembly to move.

Preferably, in the master manipulator arm capable of maintaining pose, a driven bevel gear of the bevel gear pair with orthogonal axes extends out of the upstream linkage, a cross roller bearing supporting the driven bevel gear is arranged inside the upstream linkage, and the downstream linkage is fixedly mounted on a gear shaft located at an extending-out end of the driven bevel gear.

Preferably, in the master manipulator arm capable of maintaining pose, a travel limit structure is provided at a joint between the upstream linkage and the downstream linkage for limiting a rotation angle between the upstream linkage and the downstream linkage.

Preferably, in the master manipulator arm capable of maintaining pose, the travel limit structure includes a limiting protrusion located on the upstream linkage and a limiting groove located on the downstream linkage, and the limiting protrusion and the limiting groove fitted with each other in an abutting manner.

Preferably, in the master manipulator arm capable of maintaining pose, the first joint module has a first central axis, the second joint module has a second central axis, and the third joint module has a third central axis;
the first gear pair has a fourth central axis, the second gear pair has a fifth central axis, the third gear pair has a sixth central axis, and the fourth gear pair has a seventh central axis;
extension lines of the fifth central axis, the sixth central axis, and the seventh central axis coincide at a center of a palm of the master tool; and
in an initial state, the first central axis R1, the fourth central axis R4, the fifth central axis R5 and the sixth central axis R6 coincide in an XZ plane.

A surgical robot is provided, including a surgeon console, where the master manipulator arm capable of maintaining pose according to any one of above solutions is mounted on the surgeon console.

A joint transmission structure of a manipulator arm is provided, where an upstream linkage is arranged at a front end of an arm joint of the manipulator arm, and a downstream linkage is arranged at a rear end of the arm joint; and
the arm joint is a transmission gear pair provided between the upstream linkage and the downstream linkage, and the joint transmission structure further includes a power drive device for driving the arm joint to move.

Preferably, in the joint transmission structure of the manipulator arm, the power drive device is arranged at the upstream linkage, and the power drive device includes a motor, a reducer, and an encoder that are arranged coaxially with one another; and an axial direction of the motor is perpendicular to an axis of the arm joint.

Preferably, in the joint transmission structure of the manipulator arm, the transmission gear pair includes a driving gear embracingly mounted at an output end of the reducer and a driven gear in transmission cooperation with the driving gear; and
the driving gear is arranged on the upstream linkage, and a driven gear shaft of the driven gear extends out of the arm joint and is fixedly connected to the downstream linkage.

Preferably, in the joint transmission structure of the manipulator arm, a cross roller bearing supporting the driven bevel gear is provided inside the upstream linkage, and the downstream linkage is fixedly mounted on the gear shaft of the located at an extending-out end of the driven bevel gear; and
a needle roller thrust bearing is provided at a connection between the upstream linkage and the downstream linkage to bear an axial load during axial locking of the driven gear shaft

Preferably, in the joint transmission structure of the manipulator arm, the driving gear and the driven gear are a driving bevel gear and a driven bevel gear arranged orthogonally and in transmission cooperation with each other.

Preferably, in the joint transmission structure of the manipulator arm, a driving gear shaft of the driving bevel gear is embracingly mounted on an output shaft of the reducer; and
the driving gear shaft is provided with an open slot radially running through the driving gear shaft and a locking screw for locking and mounting with the open slot.

Preferably, in the joint transmission structure of the manipulator arm, the driven gear is provided with a wire through hole, and a wire protecting sleeve is provided inside the wire through hole; and
a wire protecting plate is further provided between the wire protecting sleeve and the transmission gear pair, to isolate a wire inside the wire through hole from the transmission gear pair.

Preferably, in the joint transmission structure of the manipulator arm, the power drive device is further provided with a motor mounting seat, a mounting slide groove is provided inside the upstream linkage, and the motor mounting seat is mounted in the mounting slide groove in a slidable manner.

Preferably, in the joint transmission structure of the manipulator arm, a travel limit structure is provided at a joint between the upstream linkage and the downstream linkage for limiting a rotation angle between the upstream linkage and the downstream linkage.

A surgical robot includes a surgeon console, a master manipulator arm is mounted on the surgeon console, and an arm joint of the master manipulator arm includes the joint transmission structure of the manipulator arm according to any one of above solutions.

The master manipulator arm capable of maintaining pose according to the present application includes a front linkage proximate to a fixed mounting side and a rear linkage proximate to a master tool side, joint modules configured to adjust an angle and a displacement are arranged at first linkage joints of the front linkage, and transmission gear pairs configured to adjust an angle and a displacement are arranged at second linkage joints of the rear linkage. The master manipulator arm includes multiple linkages, where the front linkage is connected to a main body of the surgeon console, and the rear linkage extends from the front linkage and is connected to the master tool. The surgeon manipulates the master tool to transfer surgical operations from the manipulator arm to a surgical instrument. Multiple first linkage joints on the front linkage employ joint modules for angular and displacement adjustments and multiple second linkage joints on the rear linkage employ transmission gear pairs for angular and displacement adjustments. The rear linkage has a more compact mounting structure which occupies less space and is lighter in weight, and can remain at the manipulated position in case of power failure, so as to maintain the pose when the power failure occurs, thus ensuring greater stability in surgical operations.

In the joint transmission structure of a manipulator arm according to the present application, an upstream linkage is arranged at a front end of an arm joint, and a downstream linkage is arranged at a rear end of the arm joint; and the arm joint is a transmission gear pair provided between the upstream linkage and the downstream linkage, and the joint transmission structure further includes a power drive device for driving the arm joint to move. Regarding the master manipulator arm, the front linkage proximate to the fixed mounting side has great weight and volume, which needs a large torque, while the position of the rear linkage proximate to the master tool side is adjusted along with the operation of the master tool. The joint modules are arranged at linkage joints of the front linkage to adjust an angle and a displacement, and the transmission gear pairs are arranged at linkage joints of the rear linkage to adjust an angle and a displacement, and thus the angles and the displacements of the upstream linkage and the downstream linkage of each linkage joint can be adjusted, so as to be adapted to the case that the master manipulator arm includes multiple linkages, the front linkage is connected to the main body of the surgeon console, the rear linkage extends from the front linkage and is connected to the master tool, the surgeon manipulates the master tool to transfer surgical operations from the manipulator arm to the surgical instrument, the angular and displacement adjustments of the front linkage are carried out by the joint modules, the angular and displacement adjustments of the rear linkage are carried out by the transmission gear pairs, and the joint modules and the transmission gear pairs are combined for the arm joints with different load demands. In this way, the mounting structure of the upstream linkages and the downstream linkages of the rear linkage is more compact, which occupies less space and is lighter in weight, and thus the rear linkage can remain at the manipulated position in case of power failure, so as to maintain the pose when the power failure occurs, thus ensuring greater stability in surgical operations.

### BRIEF DESCRIPTION OF THE DRAWINGS

The above and other objects, features, and advantages of the present application will be clearer by the following description of the embodiments of the present application with reference to the accompanying drawings. In the drawings:

FIG. 1a is a schematic view showing a master manipulator arm capable of maintaining pose being arranged on a surgeon console according to the present application;

FIG. 1b is a schematic perspective view of FIG. 1a;

FIG. 2a is a perspective view showing the structure of the master manipulator arm capable of maintaining pose according to the present application;

FIG. 2b is a schematic rear view of FIG. 2a;

FIG. 2c is a view showing the arrangement of joint modules in the master manipulator arm of FIG. 2a;

FIG. 2d is a schematic view showing the arrangement of transmission gear pairs in FIG. 2a;

FIG. 3 is a partially enlarged view of a transmission structure of a fourth-axis linkage and a fifth-axis linkage in FIG. 2d;

FIG. 4 is a partially enlarged view of a portion A in FIG. 2d;

FIG. 5 is a schematic view of a transmission structure of a transmission gear pair between the fourth-axis linkage and the fifth-axis linkage;

FIG. 6 is a schematic view showing the mounting of a driven bevel gear of the transmission gear pair in FIG. 5; and

FIG. 7 is a schematic view of showing the mounting of a driving bevel gear of the transmission gear pair in FIG. 5.

### DETAILED DESCRIPTION OF THE EMBODIMENTS

The present application will be described hereinafter based on embodiments, but not limited to these embodiments.

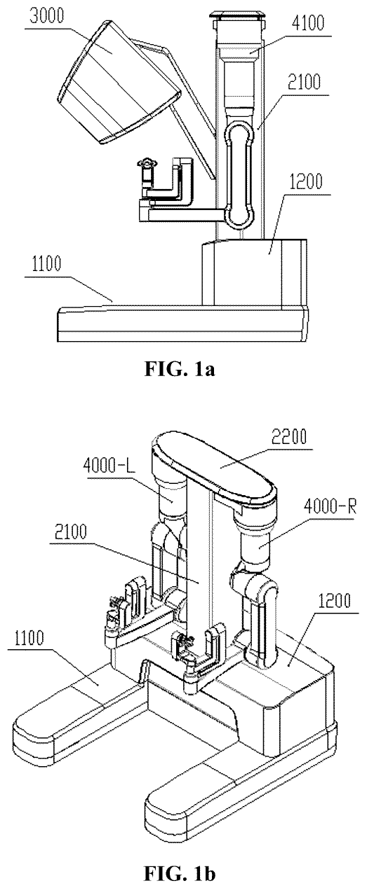

As shown in FIG. 1a and FIG. 1b, FIG. 1a is a schematic view showing a master manipulator arm capable of maintaining pose being arranged on a surgeon console according to the present application, and FIG. 1b is a schematic perspective view of FIG. 1a.

In this embodiment, a master manipulator arm capable of maintaining pose is provided, which is fixedly mounted on a surgeon console. The surgeon console includes a base 1100, an electrical box 1200, a stand column mechanism 2100, a column crossbeam component 2200, a viewfinder mechanism 3000, and a left master manipulator arm 4000-L and a right master manipulator arm 4000-R for bimanual manipulation. The surgeon console is further provided with an armrest operation panel, a foot pedal mechanism, etc. The left manipulator arm 4000-L is arranged on a left side of the surgeon console, and the right manipulator arm 4000-R is arranged on a right side of the surgeon console.

The left master manipulator arm 4000-L and the right master manipulator arm 4000-R correspond to a left-hand manipulation and a right-hand manipulation of the surgeon, respectively. The left master manipulator arm 4000-L and the right master manipulator arm 4000-R are arranged in different orientations relative to a master tool, while arrangements of linkages of the left master manipulator arm 4000-L and the right master manipulator arm 4000-R are the same. In this embodiment, the right master manipulator arm is taken as an example for structural illustration. A front linkage of the master manipulator arm is proximate to the column crossbeam component 2200 of the console. Specifically, a first-axis linkage 4100 of the front linkage is fixed to the column crossbeam component 2200 of the console by screws. The stand column mechanism has a lifting function to adjust the master tool to the most comfortable position for the surgeon's operation according to ergonomics

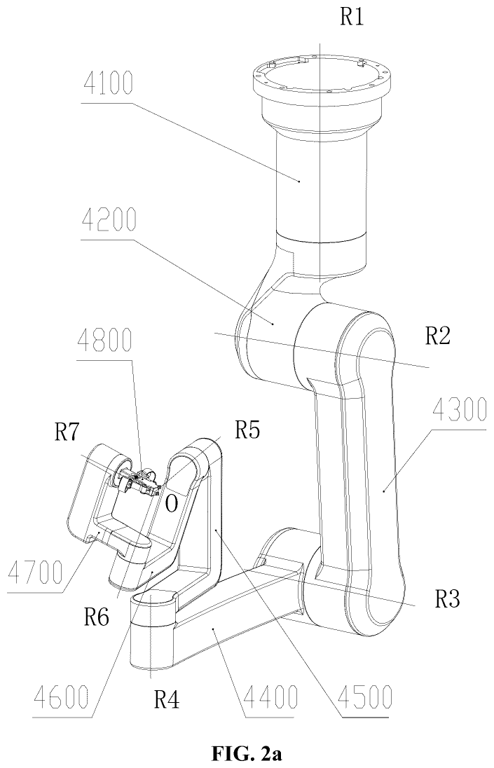

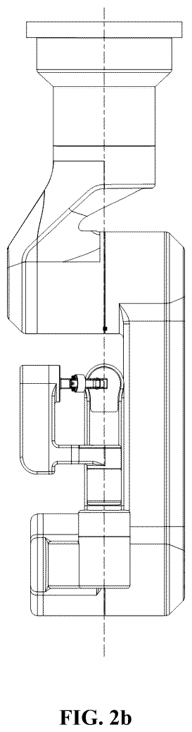

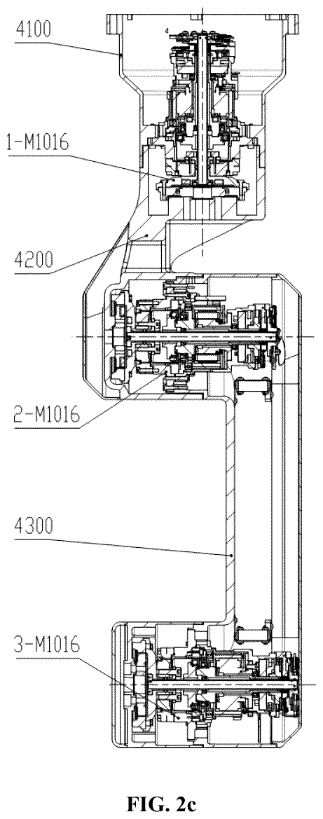

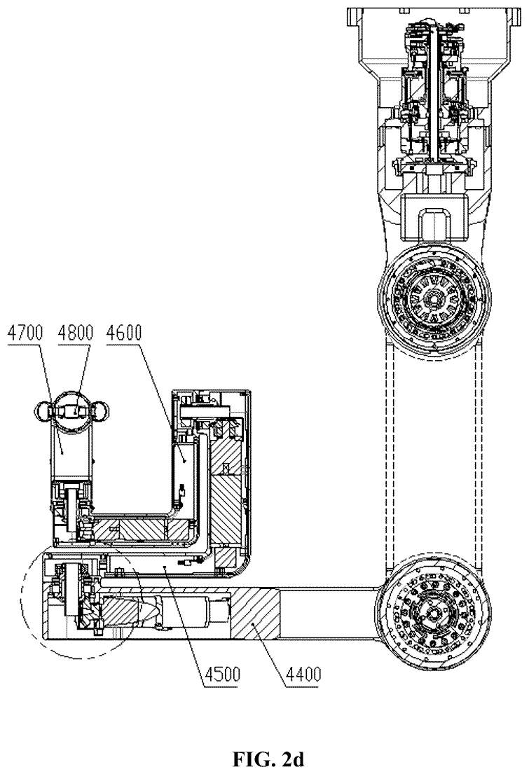

With reference to FIG. 2a to FIG. 2d, where FIG. 2a is a perspective view showing the structure of the master manipulator arm capable of maintaining pose according to the present application, FIG. 2b is a schematic rear view of FIG. 2a, FIG. 2c is a view showing the arrangement of joint modules in the master manipulator arm of FIG. 2a, and FIG. 2d is a schematic view showing the arrangement of transmission gear pairs in FIG. 2a.

In the master manipulator arm, the front linkage is connected to a main body of the surgeon console, and the rear linkage is used to support the master tool. The master manipulator arm includes the front linkage proximate to a fixed mounting side and the rear linkage proximate to a master tool side. Joint modules configured to adjust an angle and a displacement are arranged at first linkage joints of the front linkage, and transmission gear pairs configured to adjust an angle and a displacement are arranged at second linkage joints of the rear linkage.

The master manipulator arm includes multiple linkages, where the front linkage is connected to the main body of the surgeon console, and the rear linkage extends from the front linkage and is connected to the master tool. The surgeon manipulates the master tool to transfer surgical operations from the manipulator arm to a surgical instrument. Multiple first linkage joints on the front linkage employ joint modules for angular and displacement adjustments, and multiple second linkage joints on the rear linkage employ transmission gear pairs for angular and displacement adjustments. Therefore, the rear linkage has a more compact mounting structure which occupies less space and is lighter in weight, and can remain at the manipulated position in case of power failure, so as to maintain the pose when the power failure occurs, thus ensuring greater stability in surgical operations.

The master manipulator arm includes multiple linkages, where the front linkage is connected to the main body of the surgeon console, and the rear linkage extends from the front linkage and is connected to the master tool. The rear linkage is used to directly implement the displacement and the motion control of the master tool, the surgeon can manipulate the master tool to transfer surgical operations from the manipulator arm to the surgical instrument. The front linkage is directly connected to the main body of the surgeon console. In addition, the multiple first linkage joints of the front linkage employ the joint modules for angular and displacement adjustments and the multiple second linkage joints of the rear linkage employ the transmission gear pairs for angular and displacement adjustments. For each of the multiple arm joints of the rear linkage, a linkage located at a front end of the arm joint is defined as an upstream linkage, and a linkage located at a rear end of the arm joint is defined as a downstream linkage. The arm joints employ the transmission gear pairs for the angular and displacement adjustments of the upstream linkages and the downstream linkages of the rear linkage. The master manipulator arm includes multiple linkages, the front linkage is connected to the main body of the surgeon console, the rear linkage extends from the front linkage and is connected to the master tool, the surgeon manipulates the master tool to transfer surgical operations from the manipulator arm to the surgical instrument, the angular and displacement adjustments of the front linkage are carried out by the joint modules, the angular and displacement adjustments of the rear linkage are carried out by the transmission gear pairs, and the joint modules and the transmission gear pairs are combined for the arm joints with different load demands. In this way, the mounting structure of the upstream linkages and the downstream linkages of the rear linkage is more compact, which occupies less space and is lighter in weight, and thus the rear linkage can remain at the manipulated position in case of power failure, so as to maintain the pose when the power failure occurs, thus ensuring greater stability in surgical operations.

Further, the front linkage includes a first-axis linkage 4100, a second-axis linkage 4200, a third-axis linkage 4300, and a fourth-axis linkage 4400. The first-axis linkage 4100 and the second-axis linkage 4200 are connected through a first joint module 1-M1016, the second-axis linkage 4200 and the third-axis linkage 4300 are connected through a second joint module 2-M1016, and the third-axis linkage 4300 and the fourth-axis linkage 4400 are connected through a third joint module 3-M1016.

The rear linkage includes the fourth-axis linkage 4400, a fifth-axis linkage 4500, a sixth-axis linkage 4600, a seventh-axis linkage 4700, and a master tool 4800, where the master tool 4800 is located at a free end of the seven-axis linkage 4700.

The fourth-axis linkage 4400 and the fifth-axis linkage 4500 are in transmission through a first gear pair, specifically through a first bevel gear pair, and power is transferred by a first reduction motor 4416. The fifth-axis linkage 4500 and the sixth-axis linkage 4600 are in transmission through a second gear pair, and power is transferred by a second reduction motor 4516. The sixth-axis linkage 4600 and the seventh-axis linkage 4700 are in transmission through a third gear pair, and power is transferred by a third reduction motor 4616. The seventh-axis linkage 4700 and the master tool 4800 are in transmission through a fourth gear pair, and power is transferred by a fourth reduction motor 4716. The master tool 4800 is configured to perform an opening and closing movement, and a fifth reduction motor 4816 is provided in the master tool 4800.

Preferably, the first gear pair, the second gear pair, the third gear pair, and the fourth gear pair are all bevel gear pairs.

In a specific embodiment of the present application, an angle sensor and a displacement sensor are provided in each of the first joint module 1-M1016, the second joint module 2-M1016 and the third joint module 3-M1016, to record movement velocities and positions of downstream shafts and upload them to a control system.

In a specific embodiment of the present application, force sensors, configured for measuring a torque of a joint movement between the corresponding linkages, are provided in the front linkage and the rear linkage. The control system controls the manipulator arm of the surgical robot for surgical operation, so as to map the pose of the master manipulator arm to the slave manipulator arm and perceive the operating force of the slave manipulator arm through the force feedback of the master manipulator arm by corresponding computer algorithms.

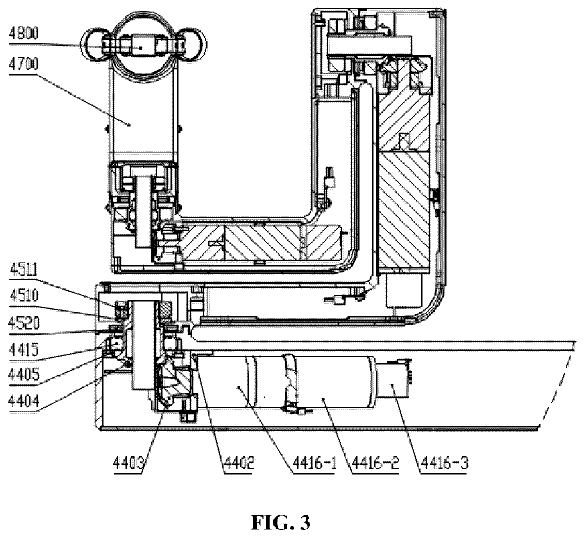

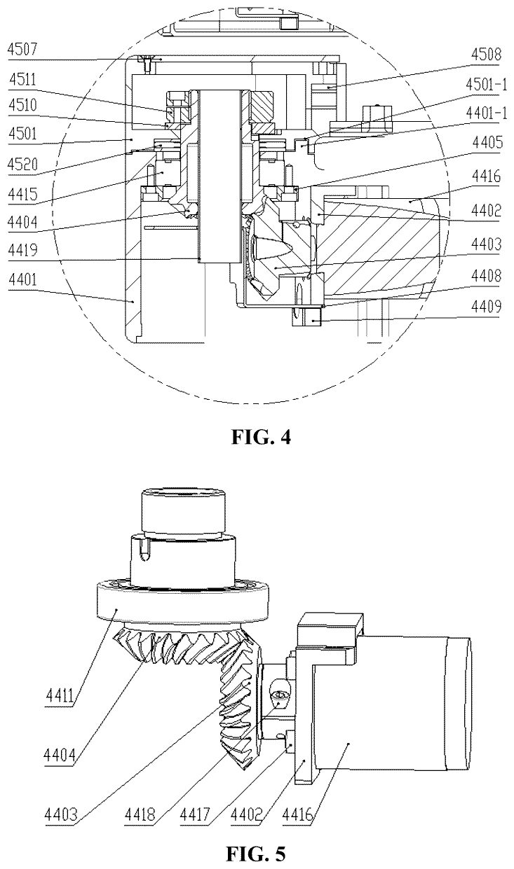

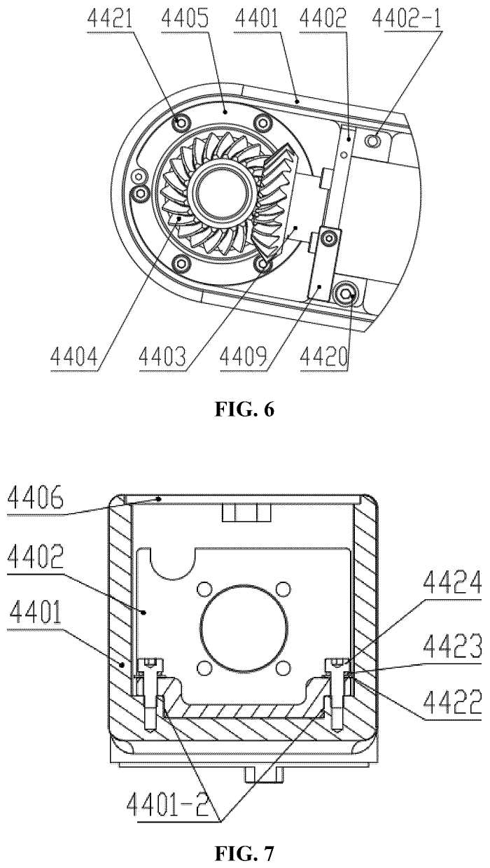

As shown in FIG. 3 to FIG. 7, FIG. 3 is a partially enlarged view of a transmission structure of a fourth-axis linkage and a fifth-axis linkage in FIG. 2d, FIG. 4 is a partially enlarged view of a portion A in FIG. 2d, FIG. 5 is a schematic view of a transmission structure of the transmission gear pair between the fourth-axis linkage and the fifth-axis linkage, FIG. 6 is a schematic view showing the mounting of a driven bevel gear of the transmission gear pair in FIG. 5, and FIG. 7 is a schematic view showing the mounting of a driving bevel gear of the transmission gear pair in FIG. 5.

In a specific embodiment of the present application, the transmission gear pair includes a reduction motor assembly located inside the upstream linkage. An axial direction of the reduction motor assembly is perpendicular to an axis of a joint located at a power output end of the reduction motor assembly. The power output end of the reduction motor assembly is provided with a bevel gear pair with orthogonal axes, which drives the downstream linkage being in transmission cooperation with the power output end of the reduction motor assembly to move.

A driven bevel gear of the bevel gear pair with orthogonal axes extends out of the upstream linkage, and a cross roller bearing supporting the driven bevel gear is provided inside the upstream linkage. The downstream linkage is fixedly mounted on a gear shaft located at an extending out end of the driven bevel gear.

A travel limit structure is provided at a joint between the upstream linkage and the downstream linkage for limiting a rotation angle between the upstream linkage and the downstream linkage. The travel limit structure includes a limiting protrusion provided on the upstream linkage and a limiting groove provided on the downstream linkage. The limiting protrusion and the limiting groove fit with each other in an abutting manner.

The first gear pair between the fourth-axis linkage 4400 and the fifth-axis linkage 4500 is taken as an example for illustration, the fourth-axis linkage 4400 is regarded as the upstream linkage and the fifth-axis linkage 4500 is regarded as the downstream linkage.

As shown in FIG. 3 and FIG. 4, a reduction motor assembly 4416 including a DC motor 4416-2, a reducer 4416-1, and an encoder 4416-3, a motor mounting seat 4402, and a driving bevel gear 4403, a driven bevel gear 4404, and a bearing retainer ring 4405 are arranged inside the fourth-axis linkage 4400. A positioning ring 4510, an axial locking nut 4511, and a needle roller thrust bearing 4520 are arranged at an end of the fourth-axis linkage connecting to the fifth-axis linkage 4500.

A reduction motor and a multi-turn absolute encoder are provided at each shaft. The axial direction of the motor is perpendicular to the axis of the joint, and a bevel gear pair with orthogonal axes is provided for transmission. The driven gear is provided with a crossed roller bearing and a needle roller thrust bearing in the axial direction, which can simultaneously bear large axial load and radial load, and thus the axial mounting length can be effectively reduced, to achieve a compact structure, a small occupied space, and a light weight.

Specifically, as shown in FIG. 4 to FIG. 6, the driven bevel gear 4404 is mounted on the crossed roller bearing 4415, and the crossed roller bearing 4415 is mounted in a bearing hole of the fourth-axis linkage body 4401 and then pressed and fixed by the bearing retainer ring 4405.

A keyway is provided on the shaft of the driven bevel gear 4404, and another keyway is provided at the fifth-axis linkage body 4501 at a position fitted with the gear shaft. The driven bevel gear 4404 and the fifth-axis linkage body 4501 are radially positioned by a positioning key on the positioning ring 4510, and then the driven bevel gear 4404 and the fifth-axis linkage body 4501 are axially positioned by the locking nut 4511. The needle roller thrust bearing 4520 is mounted at the connection between the fourth-axis linkage 4401 and the fifth-axis linkage 4501 to bear the axial load when the gear shaft is locked axially, thus increasing the radial force bearing area, and eliminating the shaking phenomenon during the operation of the manipulator arm.

A rotation travel of the joint between the fourth-axis linkage 4400 and the fifth-axis linkage 4500 is limited. A limiting protrusion 4401-1 is provided on the fourth-axis linkage 4400 and a limiting groove 4501-1 is provided on the fifth-axis linkage 4500.

The connection between the fifth-axis linkage 4500 and the sixth-axis linkage 4600 and the connection between the sixth-axis linkage 4600 and the seventh-axis linkage 4700 are the same as the connection between the fourth-axis linkage 4400 and the fifth-axis linkage 4500.

The driving bevel gear 4403 is provided with a release groove that radially extends through the driving bevel gear shaft. A threaded connection hole is formed on an outer periphery of the output shaft of the reduction motor for locking the driving bevel gear onto the output shaft of the reduction motor 4416 by a locking screw 4418. The reduction motor is fixed to the motor mounting seat 4402 by a screw 4417.

The motor mounting seat 4402 and the fourth-axis linkage body 4401 are fitted through a slide groove. In addition, a waist-shaped adjustment hole 4402-1 is formed in the motor mounting seat 4402. A fit clearance between the driving bevel gear 4403 and the driven bevel gear 4404 can be adjusted along the slide groove, and then the driving bevel gear 4403 and the driven bevel gear 4404 are locked and fixed by a screw combination 4424 (with a spring washer and a flat washer).

The driven bevel gear 4404 is mounted on the cross roller bearing 4415, the cross roller bearing 4415 is mounted in the bearing hole of the fourth-axis linkage 4401 and then pressed and fixed by the bearing cap 4405.

A hollow wire through hole is provided in the driven bevel gear 4404, where a hole diameter may be set according to the wiring demands. A wear-resistant wire protecting sleeve 4419 is provided inside the hole to prevent the wires from being worn on the gear shaft during rotation. A wire protecting plate 4408 is provided at the gear pair to prevent the wires from being twisted and collided by the gear during rotation. A wire clamp 4409 is fixedly arranged on the wire bundle to prevent the wires from being dragged and pulled during rotation.

With reference to FIG. 2a and FIG. 2b, the schematic views of the manipulator arm on the surgeon console are shown.

The second-axis linkage 4200 rotates around a first central axis R1, the third-axis linkage 4300 rotates around a second central axis R2, the fourth-axis linkage 4400 rotates around a third central axis R3, the fifth-axis linkage 4500 rotates around a fourth central axis R4, the sixth-axis linkage 4600 rotates around a fifth central axis R5, the seventh-axis linkage 4700 rotates around a sixth central axis R6, and the master tool 4800 rotates around a seventh central axis R7.

Extension lines of the fifth central axis R5, the sixth central axis R6, and the seventh central axis R7 intersect at a point 0, and a center of a palm of the master tool is also at the point 0.

When the axes are at a position which is set as the “zero” position, that is, when the master manipulator arm is in an initial state, the first central axis R1 and the second central axis R2 are in the same plane and are perpendicular to each other; the second central axis R2 is parallel to the third central axis R3; the third central axis R3 and the fourth central axis R4 are in an XZ plane and are perpendicular to each other; and the fourth central axis R4 and the fifth central axis R5 are in an YZ plane and are perpendicular to each other, and the fourth central axis R4 is parallel to the first central axis R1. Meanwhile, in the XZ plane, the fourth central axis R4 coincides with the first central axis R1. The fifth central axis R5 and the sixth central axis R6 are in the YZ plane and are perpendicular to each other. The sixth central axis R6 and the seventh central axis R7 are in the XZ plane and are perpendicular to each other.

When each axis is at the position which is set as the “zero” position, the first central axis R1, the fourth central axis R4, the fifth central axis R5, and the sixth central axis R6 coincide in the XZ plane.

Based on the master manipulator arm capable of maintaining pose provided in the above embodiments, a surgical robot is further provided in the present invention, which includes a surgeon console, where the master manipulator arm capable of maintaining pose provided in the above embodiments is mounted on the surgeon console.

In the surgical robot provided in this embodiment, the master manipulator arm has the joint transmission structure of the manipulator arm described in above embodiments.

Since the master manipulator arm maintaining the pose according to the above embodiments is employed in the surgical robot, the beneficial effects of the surgical robot achieved by the master manipulator arm capable of maintaining pose may refer to above embodiments.

Only preferred embodiments of the present invention are described above, which are not intended to limit the present invention. For those skilled in the art, the present invention may have various modifications and variations. Any modifications, equivalent substitutions, improvements, etc. made within the spirit and principle of the present invention shall be included within the scope of the present invention.

1. A master manipulator arm capable of maintaining pose, comprising:
a front linkage proximate to a fixed mounting side; and
a rear linkage proximate to a master tool side, wherein
joint modules configured to adjust an angle and a displacement are arranged at first linkage joints of the front linkage, and transmission gear pairs configured to adjust an angle and a displacement are arranged at second linkage joints of the rear linkage.

2. The master manipulator arm capable of maintaining pose according to claim 1, wherein
the front linkage comprises a first-axis linkage, a second-axis linkage, a third-axis linkage, and a fourth-axis linkage, and wherein
a first joint module of the joint modules is arranged between the first-axis linkage and the second-axis linkage, a second joint module of the joint modules is arranged between the second-axis linkage and the third-axis linkage, and a third joint module of the joint modules is arranged between the third-axis linkage and the fourth-axis linkage.

3. The master manipulator arm capable of maintaining pose according to claim 1, wherein
the rear linkage comprises the fourth-axis linkage, a fifth-axis linkage, a sixth-axis linkage, a seventh-axis linkage; and wherein
a master tool is arranged at a free end of the seventh-axis linkage;
the fourth-axis linkage and the fifth-axis linkage are in transmission through a first gear pair of the transmission gear pairs, and a first reduction motor is provided in the first gear pair;
the fifth-axis linkage and the sixth-axis linkage are in transmission through a second gear pair of the transmission gear pairs, and a second reduction motor is provided in the second gear pair;
the sixth-axis linkage and the seventh-axis linkage are in transmission through a third gear pair of the transmission gear pairs, and a fourththird reduction motor is provided in the third gear pair; and
the seventh-axis linkage and the master tool are in transmission through a fourth gear pair of the transmission gear pairs, and a fourth reduction motor is provided in the fourth gear pair.

4. The master manipulator arm capable of maintaining pose according to claim 3, wherein the first gear pair, the second gear pair, the third gear pair, and the fourth gear pair are all bevel gear pairs.

5. The master manipulator arm capable of maintaining pose according to claim 2, wherein an angle sensor and a displacement sensor are provided in each of the first joint module, the second joint module, and the third joint module, to record movement velocities and positions of joint shafts and upload the movement velocities and the positions to a control system; and
a force sensor, configured for measuring a torque of a joint movement between the corresponding linkages, is provided in each of the front linkage and the rear linkage.

6. (canceled)

7. The master manipulator arm capable of maintaining pose according to claim 5, wherein
each of the transmission gear pairs comprises a reduction motor assembly arranged inside an upstream linkage, an axial direction of the reduction motor assembly is perpendicular to an axis of a joint located at a power output end of the reduction motor assembly; and
the power output end of the reduction motor assembly is provided with a bevel gear pair with orthogonal axes, which is configured to drive a downstream linkage being in transmission cooperation with the power output end of the reduction motor assembly to move.

8. The master manipulator arm capable of maintaining pose according to claim 7, wherein
a driven bevel gear of the bevel gear pair with orthogonal axes extends out of the upstream linkage, a cross roller bearing supporting the driven bevel gear is provided inside the upstream linkage, and the downstream linkage is fixedly mounted on a gear shaft located at an extending-out end of the driven bevel gear.

9. The master manipulator arm capable of maintaining pose according to claim 8, wherein a travel limit structure is provided at a joint between the upstream linkage and the downstream linkage for limiting a rotation angle between the upstream linkage and the downstream linkage.

10. The master manipulator arm capable of maintaining pose according to claim 9, wherein the travel limit structure comprises a limiting protrusion provided on the upstream linkage and a limiting groove provided on the downstream linkage, and the limiting protrusion and the limiting groove fit with each other in an abutting manner.

11. The master manipulator arm capable of maintaining pose according to claim 4, wherein
the first joint module has a first central axis, the second joint module has a second central axis, and the third joint module has a third central axis;
the first gear pair has a fourth central axis, the second gear pair has a fifth central axis, the third gear pair has a sixth central axis, and the fourth gear pair has a seventh central axis;
extension lines of the fifth central axis, the sixth central axis, and the seventh central axis coincide at a center of a palm of the master tool; and
in an initial state, the first central axis R1, the fourth central axis R4, the fifth central axis R5 and the sixth central axis R6 coincide in an XZ plane.

12. A surgical robot, comprising a surgeon console, wherein the master manipulator arm capable of maintaining pose according to claim 1 is mounted on the surgeon console.

13. A joint transmission structure of a manipulator arm, wherein
an upstream linkage is arranged at a front end of an arm joint of the manipulator arm, and a downstream linkage is arranged at a rear end of the arm joint;
the arm joint is a transmission gear pair provided between the upstream linkage and the downstream linkage, and the joint transmission structure further comprises a power drive device for driving the arm joint to move.

14. The joint transmission structure of the manipulator arm according to claim 13, wherein the power drive device is arranged at the upstream linkage, and the power drive device comprises a motor, a reducer, and an encoder that are arranged coaxially with one another; and wherein an axial direction of the motor is perpendicular to an axis of the arm joint.

15. The joint transmission structure of the manipulator arm according to claim 14, wherein the transmission gear pair comprises a driving gear embracingly mounted at an output end of the reducer and a driven gear in transmission cooperation with the driving gear; and wherein
the driving gear is arranged on the upstream linkage, and a driven gear shaft of the driven gear extends out of the arm joint and is fixedly connected to the downstream linkage.

16. The joint transmission structure of the manipulator arm according to claim 15, wherein
the driving gear and the driven gear are a driving bevel gear and a driven bevel gear arranged orthogonally and in transmission cooperation with each other;
a cross roller bearing supporting the driven bevel gear is provided inside the upstream linkage, and the downstream linkage is fixedly mounted on the gear shaft located at an extending-out end of the driven bevel gear; and
a needle roller thrust bearing is provided at a connection between the upstream linkage and the downstream linkage to bear an axial load during axial locking of the driven gear shaft.

17. (canceled)

18. The joint transmission structure of the manipulator arm according to claim 16, wherein a driving gear shaft of the driving bevel gear is embracingly mounted on an output shaft of the reducer; and the driving gear shaft is provided with an open slot radially running through the driving gear shaft and a locking screw for locking and mounting with the open slot.

19. The joint transmission structure of the manipulator arm according to claim 18, wherein the driven gear is provided with a wire through hole, and a wire protecting sleeve is provided inside the wire through hole; and wherein
a wire protecting plate is further provided between the wire protecting sleeve and the transmission gear pair, to isolate a wire inside the wire through hole from the transmission gear pair.

20. The joint transmission structure of the manipulator arm according to claim 19, wherein the power drive device is further provided with a motor mounting seat, a mounting slide groove is provided inside the upstream linkage, and the motor mounting seat is mounted in the mounting slide groove in a slidable manner.

21. The joint transmission structure of the manipulator arm according to claim 20, wherein a travel limit structure is provided at a joint between the upstream linkage and the downstream linkage for limiting a rotation angle between the upstream linkage and the downstream linkage.

22. A surgical robot, comprising a surgeon console, wherein a master manipulator arm is mounted on the surgeon console, and an arm joint of the master manipulator arm comprises the joint transmission structure of the manipulator arm according to claim 13.
